(19) United States
(12) Patent Application Publication
(10) Pub. No.: US 2010/0168709 A1
(43) Pub. Date: Jul. 1, 2010

(54) ADJUSTABLE DIAPER COVER WITH POCKETS

(76) Inventor: Mara Jeanne HODGKIN, Temple, ME (US)

Correspondence Address: PATENT LAW OFFICE OF D.R. HASZKO;c/o Dennis R. Haszko, 499 MOSHER HILL ROAD, FARMINGTON, ME, 04938, US

(21) Appl. No.: 12/347,776

(22) Filed: Dec. 31, 2008

(51) Int. Cl. A61F 13/15

(52) U.S. Cl. 604/385.14; 604/385.22; 604/386

(57) ABSTRACT

A reusable diaper cover adapted to fit infants and toddlers of various sizes enabling the use of the same reusable cloth diaper insert. The diaper cover includes an outer waterproof layer and a corresponding inner layer of non-absorbent material. The inner layer includes an opening therein into which the cloth diaper insert is placed. The diaper insert is held in place by way of two pockets formed by the inner and outer layers at opposite ends of the opening. A configuration of fasteners is provided to enable adjustability of the diaper cover in terms of waist circumference and garment rise.

### FIELD OF THE INVENTION

The present invention relates generally to diapers. More particularly, the present invention relates to a reusable diaper cover adapted to fit infants and toddlers of various sizes enabling the use of the same reusable cloth diaper insert.

### BACKGROUND OF THE INVENTION

In general, diapers have likely been known since civilization began. Most existing diapers consist of some sort of absorbent material shaped in a manner to fit the general groin and seat area of an infant. Such diapers may be reusable or disposable. They may also be of various sizes ranging from a size suitable for the smallest of infants to the largest of toddlers. Some diapers are also suitably shaped and sized for an adult user to address adult incontinence.

Basic reusable diapers often involve an absorbent fabric that is folded generally into a triangular configuration which is adapted to be fitted about the groin and seat area of an infant. In a conventional construction, the corners of the triangular-shaped folded fabric are joined one to another by means of various types of adhesive or connecting means. One widely used means is that of a safety pin. More often in disposable diapers, one may see the use of adhesive-backed strips which are adapted to adhere, or connect to the outer surface of the diaper. In reusable diaper technology, it is more common to use various types of hook and pile fasteners such as VELCRO™ made commercially available by the Velcro Corporation of New York. However, such hook and pile fasteners may be difficult to clean if soiled.

With the mass production of diapers that utilize a large amount of paper fabric in their structure, many diapers are presently intended to be disposable. The typical disposable diaper avoids the need for the user to wash the soiled diaper, and this aspect of the diaper contributes to its ease in use. However modern and convenient these disposable diapers appear to be, the sad truth is that since their introduction in the early 1960's disposable diapers have contributed a massive amount of waste to the environment.

Many studies critical of disposable diapers point out the many problems associated with disposable diapers. Problems include that fact that many trees are cut down in their manufacture, intestinal viruses and live polio viruses from vaccines have been found in feces in disposable diapers removed from “sanitary” landfills, flushing diapers can ruin septic tanks and plumbing lines and damage sewage-treatment plants, and only commercial incinerators can safely burn disposables. Further problems include the increasing presence of such diapers improperly disposed of in parks and preservation areas or sites near underground streams or near public and private water supplies. Beyond the obvious health hazards, some studies have indicated random samplings from one particular sanitary landfill to find that disposable diapers ranged from between 16% to an astounding 32% of the garbage extracted in each sample. Some recent Environmental Protection Agency (EPA) estimates show that over 3 million tons of disposable diapers end up in landfills.

It is also believed by some that disposable diapers may also contribute more frequent and more severe diaper rash. Moreover, recent super-absorbent disposable diapers and adult incontinence pads utilize synthetic absorbent chemical products containing sodium polyacrylate cross-linked with polymers which are chemicals which can cause severe skin infections and possibly toxic shock syndrome. Still further, bleached pulp is often the primary component of disposable diapers. More frightening is that dioxin is commonly formed when paper and wood pulp are bleached, and dioxin has been associated with cancer, liver disease, miscarriage, immune-system depression, birth defects, and genetic damage in a variety of laboratory animals. Yet still further, some studies of laboratory mice have linked disposable diapers to eye, nose, and throat irritation, including bronchoconstriction similar to that of an asthma attack. Chemicals released from disposable diapers included toluene, xylene, ethylbenzene, styrene, and isopropylbenzene, among others. By contrast, cloth diapers are not found to cause respiratory problems among mice.

The good news is that “green” movements promoting recyclable and environmentally-friendly products throughout industrialized nations have begun to have an effect on the waste and ill effects generated by disposable diapers. Thus, the tide is turning as more and more people return to reusable diapers. Relatedly, many improvements have been made to ease the convenience of reusable diapers. Even so, the variety of sizes and secure placement of disposable diapers have been design hurdles not easily overcome by a reusable diaper. Indeed, many reusable diapers come in a variety of sizes where several varying sizes must be purchased over the duration of the infant to toddler to trained-child period. Still further, reusable diapers are predominantly designed such that they require a complete substantial cleaning. Some two-part reusable diapers include an absorbent cloth pad and a cover that holds the pad. However, this design as well will often include a complete cleaning of both the absorbent internal pad and the cover.

It is, therefore, desirable to provide a reusable diaper device that overcomes the difficulties of secure placement and variation of size while still being convenient, cost-effective, and environmentally-friendly.

### SUMMARY OF THE INVENTION

It is an object of the present invention to obviate or mitigate at least one disadvantage of previous reusable diapers.

In a first aspect, the present invention provides an adjustable diaper cover, the diaper cover including: an outer surface of waterproof material having an outer periphery; an inner surface of non-absorbent material, the inner surface having a first periphery shaped identically to the outer periphery of the outer surface, the inner surface and the outer surface secured to one another along at least the first periphery and the outer periphery, the inner surface including an opening having a second periphery; and anterior and posterior pockets formed between the inner surface and the outer surface, each the pocket located at opposite ends of the opening; and wherein the pockets are capable of securing a diaper insert within the opening.

In further aspect, the present invention provides an adjustable diaper cover, the diaper cover including: an outer surface of waterproof material having an outer periphery; an inner surface of non-absorbent material, the inner surface having a first periphery corresponding to the outer periphery of the outer surface, the inner surface and the outer surface secured to one another along at least the first periphery and the outer periphery, the inner surface including an opening having a second periphery; and anterior and posterior pockets formed between the inner surface and the outer surface, each the pocket located at opposite ends of the opening, the outer periphery of the outer layer and the first periphery of the inner periphery forming a generally hourglass shape such that a pair of anterior wings are formed adjacent the anterior pocket and a pair of posterior wings are formed adjacent the posterior pocket; and wherein the pockets are capable of securing a diaper insert within the opening and the outer surface and the inner surface are connected to one another at a location adjacent the anterior pocket and the pair of anterior wings. Such connection of the inner and outer surfaces may stitching therebetween or any similar connecting means.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures.

FIG. 1 is a schematic illustrating the present invention in use by a toddler-aged child.

FIG. 2 is an interior surface view of the present invention including a cloth diaper insert secured within posterior and anterior pockets.

FIGS. 3A through 3D illustrate the adjustable nature of the present invention and show the device of FIG. 2 in four possible settings from smallest to largest.

FIG. 4 is an interior view of the present invention shown in a fully opened position without any diaper insert.

FIG. 4A is identical to FIG. 4 except that the present invention is shown in a stretched position.

FIG. 5 is the opposite view of FIG. 4 and shows an exterior view of the present invention in a fully open, un-stretched position.

### DETAILED DESCRIPTION

Generally, the present invention provides an improved diaper mechanism in the form of a reusable diaper cover adapted to fit infants and toddlers of various sizes enabling the use of the same reusable cloth diaper insert. While the present invention is shown and discussed in terms of children, it should be understood that the present invention may be suitable for use in the context of adult incontinence, individuals with injuries, or handicapped individuals without straying from the intended scope of the present invention. The present invention will now be described in more detail with regard to the figures.

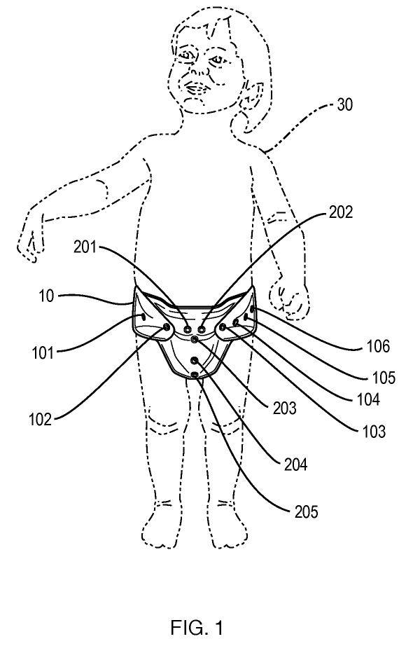

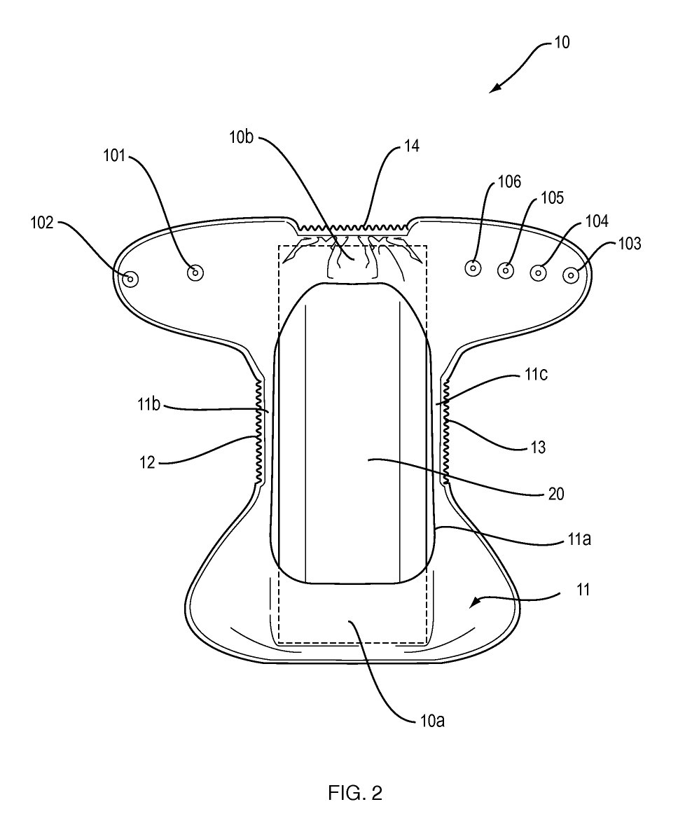

The schematic shown in FIG. 1 illustrates a diaper cover 10 in accordance with one embodiment of the present invention and in use by a toddler-aged child 30 (shown in dotted lines). From the front view shown here, fasteners 101 through 106 and 201 through 205 are visible. Such fasteners may be as shown in the form of standard push-button snaps common throughout the garment industry or may be any type of suitable fastener that would provide secure yet releasable attachment of the diaper cover 10 to the user 30. Such push-button snaps would of course be configured as either “male” or “female” snaps as deemed appropriate to one of ordinary skill would so understand. Within the diaper cover 10 is a diaper insert 20 shown and described with further reference to FIG. 2 below.

FIG. 2 shows an interior surface view of the diaper cover 10 with a diaper insert 20 in place within the diaper cover 10. For purposes herein, the diaper cover 10 with a diaper insert 20 form a complete diaper assembly. However, the present invention is drawn to the inventive diaper cover 10 for use with an appropriate diaper insert 20. The diaper insert 20 may be a standard cloth diaper or any similar absorbent material that is durable, washable, and thus suitable for use and re-use as the diaper insert 20. A useful feature of the present invention is the manner in which the diaper insert 20 is secured. The diaper insert 20 as shown is held securely at opposite ends of the diaper cover 10 through the use of an anterior pocket 10a and a posterior pocket 10b.

Each pocket 10a and 10b encompasses enough of the ends of the diaper insert 20 (as indicated by the dotted lines in FIG. 2) so that any significant movement of the diaper insert 20 is prevented. Moreover, these pockets 10a and 10b hold the diaper insert 20 in place during diaper changes and while the diaper is on the baby, while at the same time leaving the middle of the diaper in contact with the baby. The pockets 10a and 10b also prevent the wet diaper from working its way up out of the cover at the front and back and soaking the infant wearer's clothing. This innovative dual pocket design is an elegant solution that improves the overall functionality of the diaper assembly.

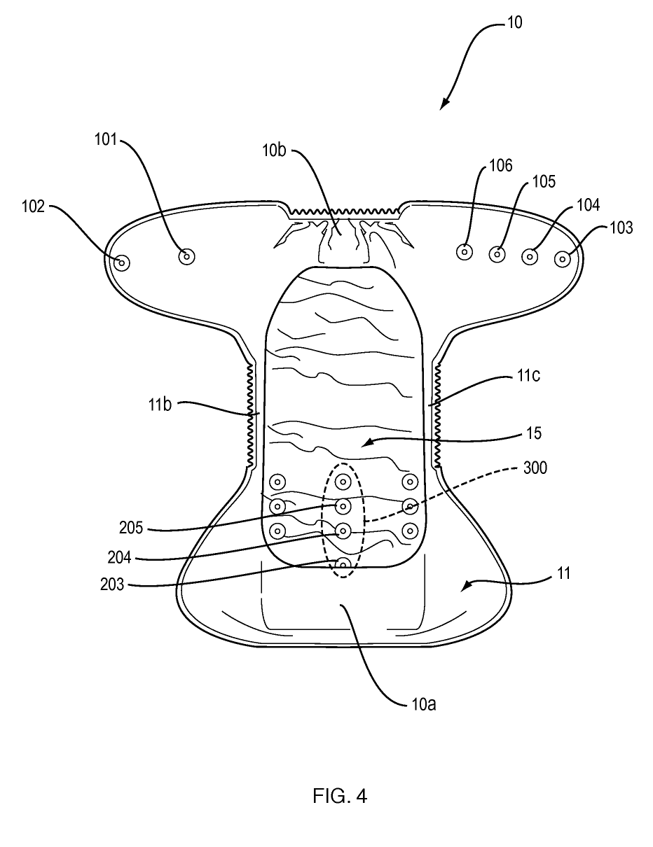

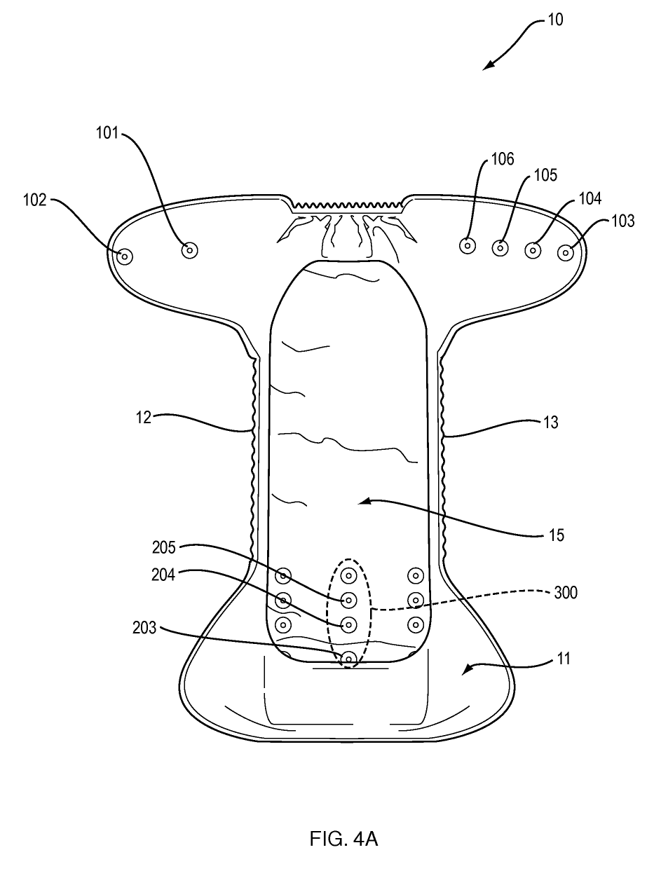

The diaper cover 20 is made from two generally hourglass-shaped pieces of material laid one over the other and sewn at their outer periphery. The sewn periphery includes elastic edging sewn to leg opening edges 12 and 13 and across the posterior edge 14 as shown. This aspect is better illustrated by way of FIG. 4A which is a view without any diaper insert and showing the elastic edgings of the leg openings stretched to their fullest extent. One layer is a waterproof outer shell and the other layer is an inner liner. As shown in FIG. 2, the inner liner 11 includes a generally oblong or rounded-rectangular opening 11a. The opening 11a provides the exposed surface of the diaper insert 20 wherein this exposed surface comes into contact with the groin and bottom areas of the user where urine and feces would gather. The diaper cover liner 11 is made of soft, non-absorbent material such as, but not limited to, suede cloth. Other soft and non-absorbent fabrics may be utilized for the liner 11 without straying from the intended scope of the present invention. However, it is important that the given material not be absorbent because such material will therefore not readily wick urine onto clothes or bedding, nor would such a material readily absorb odors.

As previously mentioned, the liner 11 lines the entire inside of the diaper cover 10 including the middle edges 11b and 11c except for the central “wet zone” where the absorbent diaper insert 20 is located. Leaving the middle open helps to keep the diaper cover 10 fresh longer because the soaked diaper is mostly in contact with the waterproof outer shell, this will not absorb any moisture or odor. Furthermore, the liner 11 which is made from non-absorbent material and placed at the middle edges 11b and 11c prevents leaks at the legs due to the liner material's non-wicking attributes. These features enable the diaper cover 10 to stay fresh longer so that it can be used many times before washing. This can significantly reduce the costs for parents because they don't need to buy as many diaper covers, nor do they need to frequently launder the diaper covers as airing out is often sufficient between launderings.

It should be readily apparent from the aforementioned description and from the inventive device as shown in FIG. 2 that the liner 11 ensures that only soft fabric is against the baby's skin rather than the non-breathable waterproof fabric of the outer shell layer. Moreover, when the diaper cover 10 is completely assembled on the infant user, the liner 11 also covers the snap fasteners such that they will not rub or press into the infant's skin.

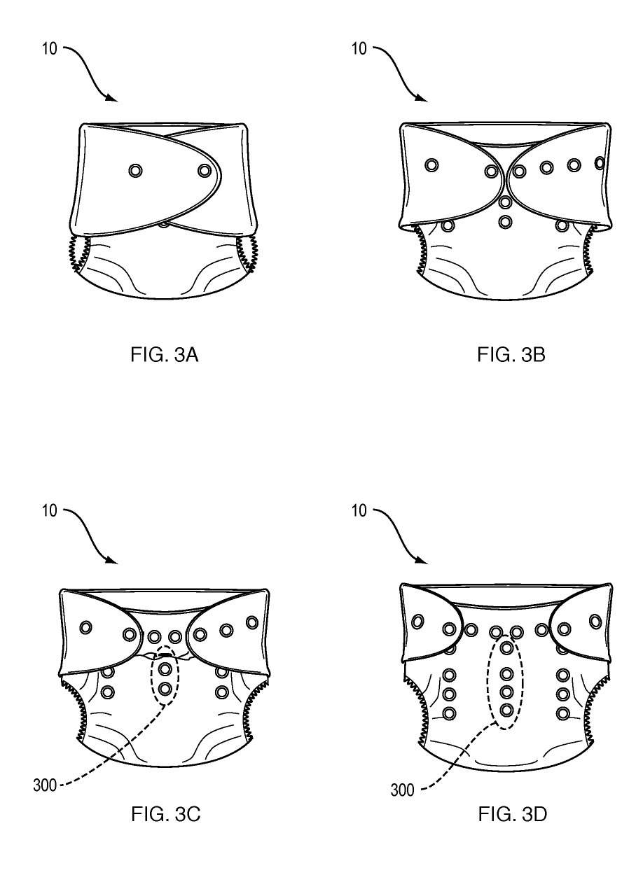

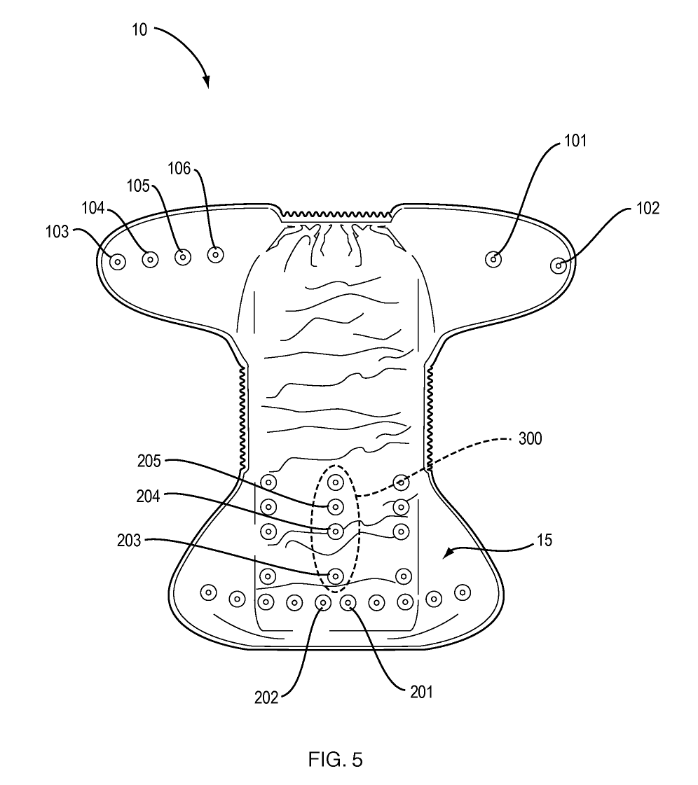

In operation, the present invention is preferably a one-size-fits-all diaper cover 10 for infants to toddlers that can utilize the same standard cloth diaper insert 20 for the range of sizes. The key to the present invention's versatility resides in the multiple fasteners. FIGS. 3A through 3D illustrate this adjustable nature of the present invention and show the device of FIG. 2 in four possible settings from smallest to largest. The diaper cover 10 accomplishes this by an arrangement of snap fasteners as seen in FIGS. 4 and 5 which respectively show the diaper cover's inner and outer surfaces with no diaper insert. For ease of use, the snap fasteners may be color coded corresponding to the four sizes illustrated in FIGS. 3A through 3D. It should further be understood that more or less than four size settings may be possible without straying from the intended scope of the present invention.

Still further, the anterior snap-down adjustment is configured such that changes in the garment rise are possible. This rise adjustability is illustrated in dotted area 300 shown in FIGS. 3C and 3D. Specifically, it can be seen that the top two snaps in dotted area 300 in FIG. 3D can be folded towards one another and snapped as seen in FIG. 3C. Although hidden from view in FIG. 3C, it should be readily apparent that the adjacent columns of four snaps each in FIG. 3D that are identical to the column shown in dotted area 300 are also concurrently snapped in the same fashion. Thus, a single diaper cover 10 can be suitable for babies 7 to 35 lbs. Accordingly, the present invention advantageously makes cloth diapering more affordable for parents because they only have to purchase one set of diaper covers, rather than requiring separately purchased covers in several sizes as your baby grows. Adjusting the garment rise as discussed above may result in some gapping of the inner and outer layers in the anterior section of the diaper cover 10. In such instance, it is optional to adhere or otherwise connect the inner and outer layers of the anterior lateral winged sections to one another. This may be accomplished, for example, by stitching a seam just outside the anterior pocket 10a—i.e., in terms of FIG. 2, such stitching would be just to the right and just to the left of anterior pocket 10a.

The present invention will be described with further reference to FIGS. 4, 4A, and 5 which illustrate the diaper cover in a fully opened (i.e., unfolded) position. Such position would be the normal position during diaper changes and during launderings. FIG. 4 corresponds to FIG. 2 except for the diaper insert 20. More particularly, the waterproof outer shell 15 can be seen in FIG. 4 as related to the non-absorbent inner surface 11 because, as shown, the diaper insert has been removed. As well, the relationship of the snaps (like-numbered in both figures) can be better viewed by the comparison of FIG. 4 to that shown in FIG. 1 where the device is fully engaged upon a user 30. FIG. 4A is identical to FIG. 4 except that the diaper cover 10 is shown stretched to illustrate the elasticity of edges 12 and 13. It should be noted also that the liner 11 may be fabricated from a different material in the leg opening areas 11b and 11c considering such design factors including, but not limited to, durability and comfort.

FIG. 5 is the opposite view of that shown in FIG. 4 and shows an exterior view of the present invention in a fully open, un-stretched position. Again, the configuration of snaps are identically numbered to those visible in FIG. 1 so as to enable one skilled in the art to fully understand the manner in which the snaps are designed to come together. Here, it is readily apparent that the vertical columns of snaps such as the column including snaps 203 through 205 enable garment rise adjustability, whereas the horizontal rows of snaps including 201, 202, and 101 through 106 enable waist adjustability. Although one specific configuration is shown, it should be clearly understood that more or less snaps (or other similar fastening mechanisms) may be provided to enable a wider range of size variation without straying from the intending scope of the present invention. Such variety of sizing would be of course important when applying the underlying invention to other uses than infant diapering such as, but not limited to adult incontinence.

As previously mentioned above, the diaper cover 20 is made from two generally hourglass-shaped pieces of material laid one over the other and sewn at their outer periphery. The inner liner 11 is an important aspect of how the diaper cover 20 is sewn. The inner liner 11 enables the use of, for example, a 1 mil polyurethane laminate (PUL). It should be understood that 2 mil PUL is also useful, though increasing the thickness beyond 2 mils may result in a stiffer and therefore less-desirable diaper cover. PUL of the 1 or 2 mil thickness is typically very soft and lightweight making the covers very comfortable. Moreover, PUL is soft on one side and polyurethane coated on the other. This makes the inner surface of the diaper cover 10 waterproof, but also smooth and plasticky. Using the preferably suede cloth inner liner 11 prevents this plasticky material from touching the baby's skin. Moreover, the configuration as shown throughout the figures results in a lightweight and flexible diaper cover having proper elasticity around the wearer's waist and at the leg openings, while the front may be snapped down to the smaller settings to vary the garment rise. The present invention enables use with prefold diapers which are very effective and at the same time inexpensive. Pairing the present invention with infant sized prefolds in combination with doublers for extra absorbency as the baby grows would enable parents to purchase only one set of covers and diapers, making the present invention the basis of an affordable diapering system as well as being easy to use, effective, and very comfortable.

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. An adjustable diaper cover, said diaper cover comprising:
an outer surface of waterproof material having an outer periphery;
an inner surface of non-absorbent material, said inner surface having a first periphery shaped identically to said outer periphery of said outer surface, said inner surface and said outer surface secured to one another along at least said first periphery and said outer periphery, said inner surface including an opening having a second periphery; and
anterior and posterior pockets formed between said inner surface and said outer surface, each said pocket located at opposite ends of said opening; and
wherein said pockets are capable of securing a diaper insert within said opening.

2. The diaper cover as claimed in claim 1 wherein said second periphery includes a continuous edge of said inner surface of non-absorbent material.

3. The diaper cover as claimed in claim 2 wherein side edges located between said anterior pocket and said posterior pocket and formed by said outer periphery and said first periphery are each elasticized.

4. The diaper cover as claimed in claim 3 wherein said outer periphery of said outer layer and said first periphery of said inner periphery form a generally hourglass shape such that a pair of anterior wings are formed adjacent said anterior pocket and a pair of posterior wings are formed adjacent said posterior pocket.

5. The diaper cover as claimed in claim 4 wherein an edge of said posterior pocket formed by said outer periphery and said first periphery is elasticized.

6. The diaper cover as claimed in claim 5 wherein said diaper cover is adjustable in circumference and rise by means of fasteners located about said outer surface.

7. The diaper cover as claimed in claim 6 wherein said fastening means are snap-fit buttons.

8. The diaper cover as claimed in claim 7 wherein at least two said snap-fit buttons are color coded according to a range of sizes.

9. The diaper cover as claimed in claim 7 wherein said outer layer is formed by a polyurethane laminate.

10. The diaper cover as claimed in claim 7 wherein said inner layer is formed by suede cloth.

11. The diaper cover as claimed in claim 7 wherein said outer surface and said inner surface are connected to one another at a location adjacent said anterior pocket and said pair of anterior wings.

12. An adjustable diaper cover, said diaper cover comprising:
an outer surface of waterproof material having an outer periphery;
an inner surface of non-absorbent material, said inner surface having a first periphery corresponding to said outer periphery of said outer surface, said inner surface and said outer surface secured to one another along at least said first periphery and said outer periphery, said inner surface including an opening having a second periphery; and
anterior and posterior pockets formed between said inner surface and said outer surface, each said pocket located at opposite ends of said opening, said outer periphery of said outer layer and said first periphery of said inner periphery forming a generally hourglass shape such that a pair of anterior wings are formed adjacent said anterior pocket and a pair of posterior wings are formed adjacent said posterior pocket; and
wherein said pockets are capable of securing a diaper insert within said opening.

13. The diaper cover as claimed in claim 12 wherein said second periphery includes a continuous edge of said inner surface of non-absorbent material.

14. The diaper cover as claimed in claim 13 wherein
an edge of said posterior pocket formed by said outer periphery and said first periphery is elasticized and
side edges located between said anterior pocket and said posterior pocket and formed by said outer periphery and said first periphery are each also elasticized.

15. The diaper cover as claimed in claim 14 wherein said diaper cover is adjustable in circumference and rise by means of fasteners located about said outer surface.

16. The diaper cover as claimed in claim 15 wherein said fastening means are snap-fit buttons.

17. The diaper cover as claimed in claim 16 wherein said snap-fit buttons are color coded according to a range of sizes.

18. The diaper cover as claimed in claim 16 wherein said outer layer is formed by a polyurethane laminate and said inner layer is formed by suede cloth.

19. The diaper cover as claimed in claim 16 wherein said outer surface and said inner surface are connected to one another at a location adjacent said anterior pocket and said pair of anterior wings.
